(19) United States
(12) Patent Application Publication
(10) Pub. No.: US 2023/0183370 A1
(43) Pub. Date: Jun. 15, 2023

(54) STEM CELL IMMUNOMODULATORY THERAPY FOR COVID-19 INFECTION

(71) Applicant: University of Florida Research Foundation, Incorporated, Gainesville, FL (US)

(72) Inventors: Duane Mitchell, Gainesville, FL (US); Catherine Flores, Gainesville, FL (US); Connor Francis, Gainesville, FL (US)

(73) Assignee: University of Florida Research Foundation, Incorporated, Gainesville, FL (US)

(21) Appl. No.: 17/916,502

(22) PCT Filed: Apr. 2, 2021
(86) PCT No.: PCT/US21/25665

§ 371 Date: Sep. 30, 2022

Related U.S. Application Data
(60) Provisional application No. 63/005,178, filed on Apr. 3, 2020.
(60) Provisional application No. 63/005,170, filed on Apr. 3, 2020.

(51) Int. Cl. C07K 16/28; A61K 35/28; A61K 9/00; A61P 31/14

(52) U.S. Cl. CPC C07K 16/2896; A61K 35/28; A61K 9/0019; A61P 31/14

(57) ABSTRACT

The disclosure provides methods of treating coronavirus infections (e.g., COVID-19) by administering hematopoietic stem cells, with or without an immune checkpoint inhibitor (e.g., PD-1 antagonist). The disclosure also provides methods of treating coronavirus infections (e.g., COVID-19) by adoptive cell transfer of polyclonal T cells and coronavirus-specific T cells (e.g., SARS-CoV-2-specific T cells).

### RELATED APPLICATIONS

This Application claims priority under 35 U.S.C. § 119(e) to U.S. provisional patent application No. 63/005,178, filed Apr. 3, 2020 and U.S. provisional patent application No. 63/005,170, filed Apr. 3, 2020, each of which is hereby incorporated by reference in its entirety.

### GOVERNMENT SUPPORT

This invention was made with government support under grant number R01 CA195563 awarded by the National Institutes of Health. The government has certain rights in the invention.

### BACKGROUND

The COVID-19 pandemic is a severe and global emergency characterized by a high penetrance in the general population and significant morbidity and mortality in the adult population across all age groups. No therapeutics have yet been proven effective for the treatment of severe illness caused by SARS-CoV-2. This is underscored by a recent randomized trial in 199 patients of lopinavir-ritonavir treatment in addition to standard of care compared to standard of care treatment alone, which showed no benefit of the anti-viral combination in hospitalized patients (Cao et al., N Engl J Med. 2020 Mar. 18. doi: 10.1056/NEJMoa2001282). There is a clear and urgent need for the development of safe and effective therapeutics for severe COVID-19.

### SUMMARY

In one aspect, the disclosure provides a method for treating a coronavirus infection (e.g., COVID-19) in a subject in need thereof, comprising administering to the subject hematopoietic stem cells in an amount effective to treat the disease. In some embodiments, the method further comprises administering to the subject a hematopoietic stem cell mobilizing agent

In one aspect, the disclosure provides a method for treating a coronavirus infection (e.g., COVID-19) in a subject in need thereof, comprising administering to the subject a hematopoietic stem cell mobilizing agent in an amount effective to treat the disease. In some embodiments, the method further comprises administering hematopoietic stem cells.

In some embodiments, the methods further comprise administering a programmed death 1 (PD-1) antagonist.

In one aspect, the disclosure provides a method for treating a coronavirus infection (e.g., COVID-19) in a subject in need thereof, comprising administering a hematopoietic stem cell mobilizing agent to the subject, harvesting hematopoietic stem cells from the subject, enriching the harvested stem cells for CCR2 positive (CCR2+), CD34 positive (CD34+), or lineage negative (lin−) cells, optionally depleting the harvested stem cells or CCR2− cells, administering to the subject the enriched harvested stem cells, and administering to the subject a PD-1 antagonist.

In some embodiments, the PD-1 antagonist is an agent that binds to and antagonizes programmed death 1 (PD-1). In some embodiments, the agent that binds to and antagonizes PD-1 is a peptide that binds PD-1. In some embodiments, the agent that binds to and antagonizes PD-1 is a humanized antibody that selectively binds PD-1. In some embodiments, the humanized antibody that selectively binds PD-1 is nivolumab, pembrolizumab, pidilizumab, MEDI-0680, REGN2810, or AMP-224. In some embodiments, the humanized antibody that selectively binds PD-1 is nivolumab.

In some embodiments, the mobilizing agent is granulocyte colony-stimulating factor (G-CSF), PEGylated G-CSF (pegfilgratism), lenogratism, a glycosylated form of G-CSF, C-X-C motif chemokine 2 (CXCL2), C-X-C chemokine receptor type 4 (CXCR-4), or plerixafor.

In some embodiments, the PD-1 antagonist is administered separately in time from or simultaneously with the hematopoietic stem cells. In some embodiments, the PD-1 antagonist is administered separately in time from or simultaneously with the hematopoietic stem cell mobilizing agent.

In some embodiments, the PD-1 antagonist is administered at the same time as, within one day of, within one week of, within one month of, within two months of, within three months of, within four months of, within five months of, or within six months of administering the hematopoietic stem cells. In some embodiments, the PD-1 antagonist is administered at the same time as, within one day of, within one week, within one month of, within two months of, within three months of, within four months of, within five months of, or within six months of administering the hematopoietic stem cell mobilizing agent.

In some embodiments, the PD-1 antagonist is administered intravenously or subcutaneously.

In some embodiments, the hematopoietic stem cells are administered intravenously.

In some embodiments, the hematopoietic stem cell mobilizing agent is administered intravenously, intradermally, or subcutaneously.

In some embodiments, the source of hematopoietic stem cells is bone marrow, bone marrow lineage depleted cells (lin−), cKit+ purified lineage negative bone marrow derived cells, Sca+ purified lineage negative bone marrow derived cells, cKit+Sca+ purified bone marrow derived cells, mobilized from host bone marrow using GM-CSF, G-CSF, mobilized from host bone marrow using AMD3100, plerixafor, or the molecule 1,1′-[1,4-phenylenebis(methylene)]bis [1,4,8,11-tetraazacyclotetradecane], umbilical cord blood or cord-blood derived stem cells, unselected umbilical cord blood stem cells (UCBSCs) or UCBSCs selected for CD34+ cells, CCR2+, or lin(−) cells, human leukocyte antigen (HLA)-matched blood, mesenchymal stem cells derived from blood or marrow, hematopoietic stem cells differentiated from induced pluripotent stem cells, mobilized peripheral blood, peripheral blood, hematopoietic stem cell subsets including lin− cells purified with CCR2+ marker, lineage negative purified peripheral blood, or CD34+ enriched peripheral blood.

In some embodiments, the source of hematopoietic stem cells is bone marrow, peripheral blood, umbilical cord blood, or induced pluripotent stem cells. In some embodiments, the source of hematopoietic stem cells is autologous. In some embodiments, the source of hematopoietic stem cells is allogeneic and the donor cells are HLA-matched to the recipient.

In some embodiments, a sample containing the hematopoietic stem cells is obtained from the subject and processed to expand the number of stem cells within the sample, in vitro, prior to administering to the subject the hematopoietic stem cells.

In some embodiments, a sample containing the hematopoietic stem cells is obtained from the subject and processed to increase the percentage of stem cells within the sample, in vitro, prior to administering to the subject the hematopoietic stem cells.

In some embodiments, at least 5, 10, 15, 20, 25, 30, 35, 40, 45, or 50 percent of the hematopoietic stem cells are CCR2 positive (CCR2+), CD34 positive (CD34+), and/or lineage negative (lin−) cells. In some embodiments, the hematopoietic stem cells for administration to the subject are enriched ex vivo for CCR2 positive (CCR2+) cells, for CD34 positive (CD34+) cells and/or for lineage negative (lin−) cells prior to administration to the subject.

In some embodiments, the coronavirus infection (e.g., COVID-19) is a severe coronavirus infection (e.g., severe COVID-19). In some embodiments, the subject is lymphopenic.

In another aspect, the disclosure provides a method for treating a coronavirus infection (e.g., COVID-19) in a subject in need thereof, comprising administering to the subject polyclonal T cells (poly-T cells) and coronavirus-specific T cells (e.g., SARS-CoV-2-specific T cells (SARS-T cells)), in amounts effective to treat the disease. In one aspect, the disclosure provides a method for treating a coronavirus infection (e.g., COVID-19) in a subject having the coronavirus infection and receiving antiviral treatment, comprising administering to the subject polyclonal T cells (poly-T cells) and coronavirus-specific T cells (e.g., SARS-CoV-2-specific T cells (SARS-T cells)) in amounts effective to treat the disease. In some embodiments, the antiviral treatment comprises administration of one or more of lopanivir, ritonavir, and remdesivir.

In some embodiments, the poly-T cells and the coronavirus-specific T cells (e.g., SARS-T cells) are autologous.

In some embodiments, the source of T cells is peripheral blood, spleen, or lymph nodes. In some embodiments, the source of T cells is peripheral blood.

In some embodiments, the poly-T cells are expanded ex vivo from T cells exposed to anti-CD3 stimulation and a cytokine milieu comprising one or more of IL-2, IL-7, IL-15, and IL-21, to drive the preferential differentiation and expansion of central and effector memory T cells.

In some embodiments, the coronavirus-specific T cells (e.g., SARS-T cells) are expanded ex vivo from T cells co-cultured with RNA electroporated antigen presenting cells expressing one or more coronavirus antigens (e.g., one or more SARS-CoV-2 antigens).

In some embodiments, the antigen presenting cells are dendritic cells, T cells, or peripheral blood mononuclear cells. In some embodiments, the antigen presenting cells are peripheral blood mononuclear cells.

In some embodiments, the coronavirus antigen is a polypeptide or immunogenic fragment thereof a structural or immunogenic component of coronavirus. In some embodiments, the coronavirus antigen (e.g., SARS-CoV-2 antigen) is a polypeptide, or an immunogenic fragment thereof, selected from the group consisting of: spike polypeptides, membrane polypeptides, and envelope polypeptides.

In some embodiments, the methods further comprise administering one or more of IL-7, IL-2, IL-15, or IL-21. In some embodiments, the methods further comprise administering IL-7. In some embodiments, the IL-7 is human recombinant IL-7.

In some embodiments, one or more of IL-7, IL-2, IL-15, or IL-21 are administered separately in time from or simultaneously with the poly-T cells and/or the coronavirus-specific T cells (e.g., SARS-T cells). In some embodiments, one or more of IL-7, IL-2, IL-15, or IL-21 are administered at the same time as, within one day of, within one week of, within one month of, within two months of, within three months within three months of, within four months of, within five months of, or within six months of administering the poly-T cells and/or the coronavirus-specific T cells (e.g., SARS-T cells).

In some embodiments, IL-7, IL-2, IL-15, or IL-15 is administered intravenously, intradermally, or subcutaneously.

In some embodiments, the one or more antiviral agents are administered separately in time from or simultaneously with the poly-T cells and/or the coronavirus-specific T cells (e.g., SARS-T cells) and/or cytokines selected from IL-7, IL-2, IL-15 and IL-21.

In some embodiments, the one or more antiviral agents are administered at the same time as, within one day of, within one week of, within one month of, within two months of, within three months of, within four months of, within five months of, or within six months of administering the poly-T cells and/or the coronavirus-specific T cells (e.g., SARS-T cells) and/or cytokines selected from IL-7, IL-2, IL-15 and IL-21.

In some embodiments, the coronavirus infection (e.g., COVID-19) is a severe coronavirus infection (e.g., severe COVID-19). In some embodiments, the subject is lymphopenic.

In some embodiments, an effect of the treatment on the disease is assessed by measuring in vivo clonal expansion of SARS-T cells.

### BRIEF DESCRIPTION OF DRAWINGS

FIG. 1 shows results of experiments on tumor bearing mice that received HSCs and PD-1 blockade, singly, or in combination. HSCs+PD-1 blockade is effective in lymphopenic mice. Prior to treatment, lymphopenia was induced in cohorts of mice by using 5Gy total body irradiation.

FIG. 2 shows effects of HSCs+PD-1 blockade on T cell expansion. The combination of HSCs+PD-1 blockade causes rapid peripheral T cell expansion.

FIG. 3 shows effects of HSCs on lymphopenic hosts. HSCs induce homeostatic cytokines in lymphopenic hosts.

FIG. 4 shows effects of HSCs+PD-1 blockade on systemic T cell immunity. The combination of HSCs+PD-1 blockade enhances systemic T cell immunity. The gene expression in the periphery (splenocytes) of treated mice was measured by qPCR array.

FIG. 5 shows a treatment scheme for nivolumab in combination with HSC transfer.

FIG. 6 shows a treatment scheme for adoptive cell transfer of polyclonal T cells and SARS-CoV-2 specific T cells.

### DETAILED DESCRIPTION

The following detailed description is made by way of illustration of certain aspects of the disclosure. It is to be understood that other aspects are contemplated and may be made without departing from the scope or spirit of the present disclosure. The following detailed description, therefore, is not to be taken in a limiting sense. Scientific and technical terms used herein have meanings commonly used in the art unless otherwise specified. The definitions provided herein are to facilitate understanding of certain terms used frequently herein and are not meant to limit the scope of the present disclosure.

The present disclosure relates to immunomodulatory therapies. In some aspects, the present disclosure relates to immunomodulatory therapies for coronavirus infections. In some aspects, the present disclosure relates to immunomodulatory therapies for sepsis.

Subject. “Subject,” used interchangeably with “patient,” means a mammal, such as a human, a nonhuman primate, a dog, a cat, a sheep, a horse, a cow, a pig, a mouse, a rat, a rodent, or a goat. In some embodiments, the subject and mammal is a human.

In some embodiments, a subject in need of treatment has one or more symptoms of a coronavirus infection. Symptoms of a coronavirus infection include, but are not limited to, fever, chills, cough, shortness of breath or difficulty breathing, fatigue, muscle or body aches, headaches, new loss of taste or smell, sore throat, congestion, runny nose, nausea, vomiting, diarrhea, pink eye (conjunctivitis), rash, pain or pressure in the chest, new confusion, inability to wake or stay awake, and pale, gray, or blue-colored skin, lips, or nail beds. In some embodiments, the subject has been diagnosed as having a coronavirus infection (e.g., using a nucleic acid assay, for example, a PCR-based assay, and/or a protein detection assay).

In further embodiments, a subject in need of treatment has one or more symptoms of sepsis. Symptoms of sepsis include, but are not limited to rapid breathing (respiratory rate ≥22 breaths per minute), high heart rate, low blood pressure (e.g., systolic blood pressure ≤100 mm Hg), shortness of breath, confusion or disorientation, extreme pain or discomfort, fever, chills, clammy or sweaty skin, patches of discolored skin, decreased urination, changes in mental ability, low platelet count, abnormal heart functions, unconsciousness, and change in mental status. In some embodiments, the sepsis is associated with the coronavirus infection. In some embodiments, the sepsis is associated with one or more other infections (e.g., viral, bacterial, fungal, protozoan, cestode, or nematode).

Treatment. “Treat”, “treating”, “treatment”, and “therapy” encompass an action that occurs while a subject is suffering from a condition which reduces the severity of the condition (or a symptom associated with the condition) or retards or slows the progression of the condition (or a symptom associated with the condition). This is therapeutic treatment.

Effective Amount. Subjects are treated with effective amounts of the solutions of the disclosure. An “effective amount” of an agent generally refers to an amount sufficient to elicit the desired biological response, i.e., treat the condition. As will be appreciated by those of ordinary skill in this art, the effective amount of an agent described herein may vary depending on such factors as the condition being treated, the mode of administration, and the age, body composition, and health of the subject.

For therapeutic treatment, an effective amount is an amount sufficient to provide a therapeutic benefit in the treatment of a condition or to reduce or eliminate one or more symptoms associated with the condition. This may encompass an amount that improves overall therapy, reduces or avoids symptoms or causes of the condition, or enhances the therapeutic efficacy of another therapeutic agent. An effective amount may be such an amount which halts the development of, inhibits the progression of, reverses the development of, or otherwise reduces or ameliorate one or more symptoms of COVID-19. In addition, an effective amount may be such an amount which slows, halts or reverses the proliferation of the coronavirus (e.g., SARS-CoV-2) in the subject.

Coronavirus infections. The present disclosure provides methods for treating coronavirus infections. In some embodiments, the coronavirus is a human coronavirus (e.g., SARS-CoV, HCoV NL63, HKU1, MERS-CoV, SARS-CoV-2, etc.). In some embodiments, the infection is caused by a human coronavirus, and includes, but is not limited to, SARS, MERS, and COVID-19. In some embodiments, the coronavirus infection is COVID-19. In some embodiments, the coronavirus infection is severe COVID-19. In some embodiments, the subject being treated for a coronavirus infection (e.g., COVID-19) is lymphopenic. In some embodiments, the subject has sepsis. In some embodiments, the lymphopenic subject has a lymphocyte count of less than about 1000 cells/μL, less than about 900 cells/μL, less than about 800 cells/μL, less than about 700 cells/μL, less than about 600 cells/μL, less than about 500 cells/μL, or less than about 400 cells/μL.

### Stem Cell Immunomodulatory Therapy

In one aspect, the present disclosure provides a stem cell immunomodulatory therapy for coronavirus infections. In one aspect, the disclosure provides methods for treating coronavirus infections by administering to a subject in need thereof hematopoietic stem cells (HSCs). In another aspect, the disclosure provides methods for treating coronavirus infections by administering to a subject in need thereof an HSC mobilizing agent. In some embodiments, the HSCs are administered in combination with an HSC mobilizing agent. In some embodiments, the HSCs and/or HSC mobilizing agent are administered in further combination with an immune checkpoint inhibitor. In some embodiments, the immune checkpoint inhibitor is a programmed death-1 (PD-1) antagonist. In some embodiments, the coronavirus infection is COVID-19, caused by the novel coronavirus, severe acute respiratory syndrome coronavirus 2 (SARS-CoV-2). In some embodiments, the coronavirus infection is severe COVID-19. In some embodiments, the subject is lymphopenic. In some embodiments, the subject has sepsis. In some embodiments, the methods of the disclosure treat symptoms of the immune response in the patient. The present disclosure also contemplates further administering one or more antiviral agents in combination with HSCs, HSC mobilizing agent and/or immune checkpoint inhibitor. The one or more antiviral agents may include, but are not limited to, lopanivir, ritonavir, and remdesivir.

In one aspect, the disclosure provides methods for treating sepsis by administering to a subject in need thereof HSCs. In another aspect, the disclosure provides methods for treating sepsis by administering to a subject in need thereof an HSC mobilizing agent. In some embodiments, the HSCs are administered in combination with an HSC mobilizing agent. In some embodiments, the HSCs and/or HSC mobilizing agent are administered in further combination with an immune checkpoint inhibitor. In some embodiments, the immune checkpoint inhibitor is a programmed death-1 (PD-1) antagonist. In some embodiments, the sepsis is associated with COVID-19, caused by SARS-CoV-2. In some embodiments, the sepsis associated with another infectious agent (e.g., a bacterial infection, a fungal infection, a protozoan infection, a cestode infection, a nematode infection, or a viral infection other than SARS-CoV-2). In some embodiments, the subject is lymphopenic. In some embodiments, the methods of the disclosure treat symptoms of the immune response in the patient. In some embodiments, the methods of the disclosure do not treat the underlying infection. In some embodiments, the methods of the disclosure treat inflammation associated with sepsis. In some embodiments, the methods of the disclosure further comprise administering one or more anti-inflammatory or antimicrobial agents in combination with HSCs, HSC mobilizing agent and/or PD-1 antagonist administration. In some embodiments, the antimicrobial agent is an antibacterial agent. In some embodiments, the antimicrobial agent is an antifungal agent. In some embodiments, the antimicrobial agent is an antiviral agent (e.g. lopanivir, ritonavir, and remdesivir). Anti-inflammatory and antimicrobial agents are known in the art.

Recent studies from patients with COVID-19 in Wuhan, China, revealed that patients who succumb to this infection are characterized by lymphopenia early in the course of hospitalization (lymphocyte counts below 800 cells/microliter) and a respiratory failure pattern called acute respiratory distress syndrome (ARDS) and/or septic shock (Zhou et al., Lancet 395:1054-1062 (2020)). The studies indicate that an uncontrolled inflammatory syndrome combined with immune suppression due to low lymphocyte counts are major contributors of mortality with COVID-19 infections. Severe and lethal COVID-19 infections are characterized by deficits in T cell immunity (lymphopenia, increased PD-1 expression and other markers of exhaustion, and loss of T cell function) (Zheng et al., Cell Mol Immunol., 2020 Mar. 17. doi: 10.1038/s41423-020-0401-3). Patients with poor outcomes from other infectious causes of sepsis are also characterized by severe impairment of T cell immunity, lymphopenia, and upregulation of the PD-1/PD-L1 pathway. Immune checkpoint blockade with PD-1/PD-L1 inhibitors has been explored as a modality to restore T cell function in septic patients and shown to be safe (Hotchkiss et al., Intensive Care Med 45:1360-1371 (2019); Hotchkiss et al., 47(5): Crit Care Med. 47(5):632-642 (2019)).

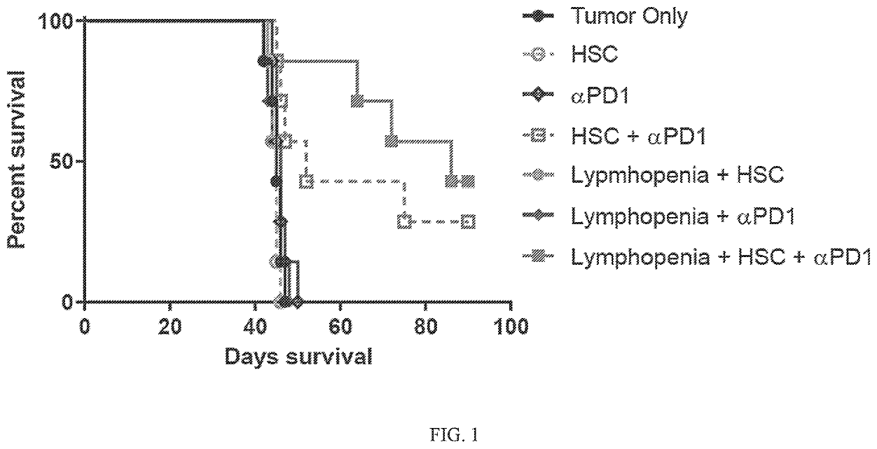

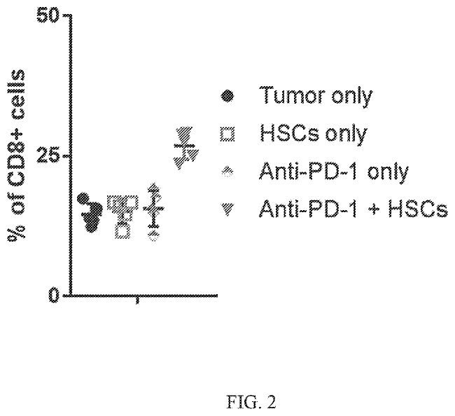

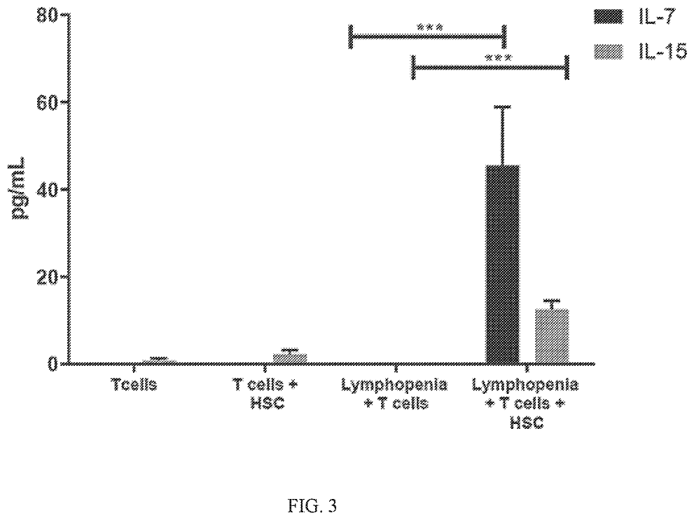

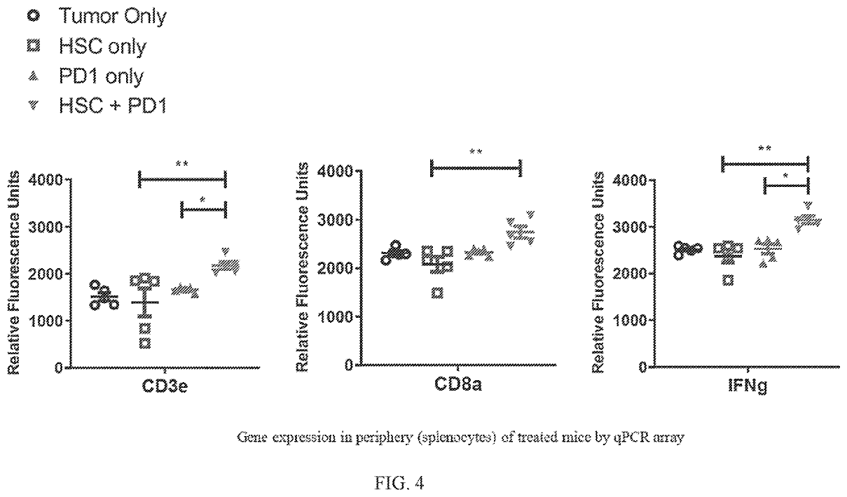

The inventors have made the observation that based on a novel mechanism of action that leads to rapid expansion of peripheral blood lymphocytes and marked enhancement of T cell function, the immunomodulatory stem cell approach described herein (e.g., administration of HSCs), combined with immune checkpoint blockade (e.g., PD-1 blockade), will safely and rapidly induce effective adaptive immune reconstitution in lymphopenic patients with COVID-19. The immunomodulatory stem cell approach described herein will also induce adaptive immune reconstitution in patients with sepsis due to other infectious causes. This observation is supported, in part, by data showing that the combination of HSCs and PD-1 blockade is (i) effective at improving survival in lymphopenic mice (FIG. 1); (ii) is able to cause rapid peripheral T cell expansion (FIG. 2); (iii) enhances systemic T cell immunity (FIG. 4). HSCs are also able to induce homeostatic cytokines in lymphopenic hosts (FIG. 3).

In some embodiments, the methods of the disclosure induce a systemic reconstitution of the immune system. In some embodiments, the methods of the disclosure induce an expansion of lymphocytes. In some embodiments, the methods of the disclosure reprogram the immune microenvironment. In some embodiments, the methods of the disclosure induce activation of tumor infiltrating lymphocytes.

Immune checkpoint inhibitors. In one aspect, the present disclosure posits that combining immune checkpoint blockade with HSCs and/or an HSC mobilizing agent enhances treatment efficacy in a subject having a coronavirus infection (e.g., COVID-19). Accordingly, in some embodiments, a method for treating a coronavirus infection (e.g., COVID-19) comprises administering HSCs and/or an HSC mobilizing agent in combination with an immune checkpoint inhibitor. In some embodiments, the immune checkpoint inhibitor is a PD-1 antagonist.

Programmed Death 1 (PD-1). In humans, programmed cell death protein 1 (PD-1) is encoded by the PDCD1 gene. PDCD1 has also been designated as CD279 (cluster of differentiation 279). This gene encodes a cell surface membrane protein of the immunoglobulin superfamily. PD-1 is a 288 amino acid cell surface protein molecule. PD-1 is expressed on the surface of activated T cells, B cells, and macrophages. PD-1 is expressed in pro-B cells and is thought to play a role in their differentiation. See T. Shinohara et al., Genomics 23 (3): 704-6 (1995). PD-1 is a member of the extended CD28/CTLA-4 family of T cell regulators (Y. Ishida et al., EMBO J. 11 (11): 3887-95, (1992)). PD-1 may negatively regulate immune responses. PD-1 limits autoimmunity and the activity of T cells in peripheral tissues at the time of an inflammatory response to infection.

PD-1 has two ligands, PD-L1 and PD-L2, which are members of the B7 family. PD-L1 protein is upregulated on macrophages and dendritic cells (DC) in response to LPS and GM-CSF treatment, and on T cells and B cells upon TCR and B cell receptor signaling, whereas in resting mice, PD-L1 mRNA can be detected in the heart, lung, thymus, spleen, and kidney.

Programmed Death 1 (PD-1) antagonists. A PD-1 antagonist, as used herein is a molecule that binds to PD-1 protein or to a gene or nucleic acid encoding PD-1 protein and inhibits or prevents PD-1 activation. Without wishing to be bound by theory, it is believed that such molecules reduce or block the interaction of PD-1 with its ligand(s) PD-L1 and/or PD-L2.

PD-1 activity may be interfered with by antibodies that bind selectively to and block the activity of PD-1. The activity of PD-1 can also be inhibited or blocked by molecules other than antibodies that bind PD-1. Such molecules can be small molecules or can be peptide mimetics of PD-L1 and PD-L2 that bind PD-1 but do not activate PD-1. Molecules that antagonize PD-1 activity include those described in U.S. Publications 20130280265, 20130237580, 20130230514, 20130109843, 20130108651, 20130017199, and 20120251537, 2011/0271358, EP 2170959B1, the entire disclosures of which are incorporated herein by reference. See also M. A. Curran, et al., Proc. Natl. Acad. Sci. USA 107, 4275 (2010); S. L. Topalian, et al., New Engl. J. Med. 366, 2443 (2012); J. R. Brahmer, et al., New Engl. J. Med. 366, 2455 (2012); and D. E. Dolan et al., Cancer Control 21, 3 (2014), all incorporated by reference herein, in their entireties. Herein, exemplary PD-1 antagonists include: nivolumab, also known as BMS-936558, OPDIVO® (Bristol-Meyers Squibb, and also known as MDX-1106 or ONO-4538), a fully human IgG4 monoclonal antibody against PD-1; pidilizumab, also known as CT-011 (CureTech), a humanized IgG1 monoclonal antibody that binds PD-1; MK-3475 (Merck, and also known as SCH 900475), an IgG4 antibody that binds PD-1; and pembrolizumab (Merck, also known as MK-3475, lambrolizumab, or KEYTRUDA®), a humanized IgG4-kappa monoclonal antibody that binds PD-1; MEDI-0680 (AstraZeneca/MedImmune), a monoclonal antibody that binds PD-1; and REGN2810 (Regeneron/Sanofi), a monoclonal antibody that binds PD-1. Another exemplary PD-1 antagonist is AMP-224 (Glaxo Smith Kline and Amplimmune), a recombinant fusion protein composed of the extracellular domain of the PD-1 ligand programmed cell death ligand 2 (PD-L2) and the Fc region of human IgG1, that binds to PD-1. Agents that interfere with the DNA or mRNA encoding PD-1 also can act as PD-1 inhibitors. Examples include a small inhibitory anti-PD-1 RNAi, an anti-PD-1 antisense RNA, or a dominant negative protein. PDL-2 fusion protein AMP-224 (co-developed by Glaxo Smith Kline and Amplimmune) is believed to bind to and block PD-1. In some embodiments, a PD-1 antagonist (e.g., an anti-PD-1 antibody) may be used for treatment in combination with hematopoietic stem cell (HSC) transfer in further combination with additional immune checkpoint blockade.

Hematopoietic Stem Cells. A hematopoietic stem cell (HSC), also called a blood stem cell, is an immature cell found in the blood and the bone marrow that can renew itself, and that can differentiate into a variety of specialized cells, such as blood and immune cells, including white blood cells, red blood cells, and platelets. HSCs can mobilize out of the bone marrow into circulating blood. HSCs facilitate constant renewal of blood cells, producing billions of new blood cells each day.

Hematopoietic Stem Cell Transplantation (HSCT). Hematopoietic stem cell transplantation (HSCT or HSC transfer) is the transplantation of HSCs, usually derived from peripheral blood, bone marrow, or umbilical cord blood. Two types of HSCT may be used in a subject: autologous stem cell transplantation, wherein the subject's own stem cells are used, or allogenic stem cell transplantation, wherein a donor's stem cells, that are genetically similar and HLA-matched to the recipient, are transplanted into the subject. In some embodiments, autologous stem cells are used for HSCT. In some embodiments, HLA-match allogenic stem cells are used for HSCT.

In autologous HSCT, a sample containing stem cells are removed from the subject, stored, and later transplanted back into the subject. HSCs represent a small fraction of the total population of blood cells in the sample, so it may be advantageous to increase the number of HSCs before administering them to the subject for treating a coronavirus infection. In some embodiments, hematopoietic stem cells are collected and expanded, before transplanting them into the subject for treatment. In some embodiments, hematopoietic stem cells are collected, expanded, and selected for from the sample, before transplanting them into the subject for treatment.

In some embodiments, stem cells can be enriched in the material used for transplantation. In some embodiments, the enrichment can occur by selectively stimulating the growth/expansion of stem cells versus other cells collected from a subject. In another embodiment, the stem cells can be enriched by isolating stem cells from other cells collected from a subject. Such selection may be so-called positive selection or negative selection. In some embodiments, in positive selection, stem cells are isolated based on the markers CCR2+, CD34+, and/or lin−, thereby enriching the HSCs for the positive marker(s). In negative selection, cells that are not stem cells are identified and removed based on markers on such other cells, leaving behind stem cells. In some embodiments, in negative selection, stem cells are isolated based on the marker CCR2−. In the negative selection, the HSCs are processed ex vivo to deplete the CCR2− cells thereby enriching the HSCs for the positive marker(s) CCR2+, CD34+, and/or lin− before administering the HSCs to the subject. Such selection procedures are well known to those of ordinary skill in the art and include but are not limited to flow cytometric analysis, microbead-based isolation, adherence assays, and/or a ligand-based selection. In some embodiments, the ligand-based selection, is based on the presence of a CCR2 ligand, e.g., CCL2. In some embodiments, the enriched HSCs may be proliferated in vitro before administration to the subject. In some embodiments, the enriched HSCs may be proliferated in vitro, and again positively selected for CCR2+, CD34+, and/or lin−, before administration to the subject. In some embodiments, the enriched HSCs may be proliferated in vitro, and negatively selected for CCR2− cells, wherein the CCR2− cells are again depleted before administering the HSCs to the subject. In some embodiments, after depletion of the CCR2− cells, less than 20% of starting population of CCR2− HSCs remain. In some embodiments, after depletion of the CCR2− cells, less than 15%, 10%, 5%, less than 2% and even less than 1% of starting population of CCR2− HSCs remain. In some embodiments, depleting CCR2− cells before administration of the HSCs to the subject results in HSCs for administration that contain no more than 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, or 80% CCR2− HSCs. In some embodiments, after positive selection for CCR2+, CD34+, and/or lin− cells; after positive selection for CCR2+, CD34+, and/or lin− cells and proliferation of the positively selected cells; or after positive selection for CCR2+, CD34+, and/or lin− cells, proliferation of the positively selected cells, and a second positive selection for CCR2+, CD34+, and/or lin− cells, and before administration of the HSCs, the HSCs for administration contain at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, or 98% CCR2+, CD34+, and/or lin− HSCs.

Sources of hematopoietic stem cells herein include: bone marrow lineage depleted cells (lin−), cKit+ purified lineage negative bone marrow derived cells, Sca+ purified lineage negative bone marrow derived cells, cKit+Sca+ purified bone marrow derived cells, mobilized from host bone marrow using G-CSF, mobilized from host bone marrow using AMD3100, plerixafor, or the molecule 1,1′-[1,4-phenylenebis(methylene)]bis [1,4,8,11-tetraazacyclotetradecane], umbilical cord blood or cord-blood derived stem cells, unselected umbilical cord blood stem cells (UCBSCs) or UCBSCs selected for CD34+ cells, CCR2+, or lin(−) cells, human leukocyte antigen (HLA)-matched blood, mesenchymal stem cells derived from blood or marrow, hematopoietic stem cells differentiated from induced pluripotent stem cells, mobilized peripheral blood, peripheral blood, hematopoietic stem cell subsets including Lin− cells purified with CCR2+ marker, lineage negative purified peripheral blood, or CD34+ enriched peripheral blood. In some embodiments, the source of HSCs is bone marrow. In some embodiments, the source of HSCs is unselected umbilical cord blood stem cells (UCBSCs) or UCBSCs selected for CD34+cells, CCR2+, or lin(−) cells. In some embodiments, the source of HSCs is autologous or allogeneic, optionally wherein, the source is bone marrow, peripheral blood, umbilical cord blood (e.g., UCBSCs or UCBSCs selected for CD34+ cells, CCR2+, or lin(−) cells) or induced pluripotent stem cells.

Hematopoietic Stem Cell Mobilizing Agent. In some embodiments, a hematopoietic stem cell mobilizing agent is administered to the subject (e.g., alone or combination with HSCs and/or an immune checkpoint inhibitor (e.g., a PD-1 antagonist)). HSC mobilization refers to the recruitment of HSCs from the bone marrow of a subject into the peripheral blood of the subject. HSC mobilizing agents include, but are not limited to, granulocyte colony-stimulating factor (G-CSF), PEGylated G-CSF (pegfilgratism), lenogratism, a glycosylated form of G-CSF, C-X-C motif chemokine 2 (CXCL2), C-X-C chemokine receptor type 4 (CXCR-4), and plerixafor. In some embodiments, an HSC mobilizing agent is administered alone.

Dosages. An exemplary effective amount of hematopoietic stem cells for injection is about 2×10⁶ cells per kilogram (kg) body weight of the subject. Exemplary effective amounts of hematopoietic stem cells for injection can range above and below this amount. Examples include from about 1, 1.5, 2, 2.5, 3, 3.5, 4, 4.5, 5, 5.5, 6, 6.5, or 7×10⁶ cells/kg.

Effective amounts of agents as used in the art range as follows: Anti-PD-1 Antibodies: 0.01 mg/kg to 20 mg/kg every 1-4 weeks. In some embodiments, such administration is for so long as the coronavirus infection persists. In some embodiments, the administration can be, for example, up to 156 weeks. In some embodiments, pembrolizumab can be administered at 10 mg/kg every two weeks, 10 mg/kg every three weeks, or 2 mg/kg every three weeks, for example, up to 96 weeks; nivolumab can be administered at 0.1 to 10 mg/kg every two weeks for example, up to 96 weeks; pidilizumab can be administered at 0.1 to 10 mg/kg every one week, 0.1 to 10 mg/kg every two weeks, or 0.1 to 10 mg/kg every three weeks, for example, up to 96 weeks.

Mobilizing Agents: Such agents are given in amounts sufficient to mobilize stem cell from bone marrow into peripheral blood. Such amounts for particular mobilizing agents have been, for example: 1 μg/kg to 20 μg/kg G-CSF per day, preferably, 5 μg/kg or 10 μg/kg G-CSF per day; 1 to 20 mg PEGylated G-CSF, preferably 6 mg or 12 mg PEGylated G-CSF; 1 to 20 μg/kg PEGylated G-CSF per day; 1 to 20 μg/kg lenogratism per day; 1 to 40 μg/m² C-X-C chemokine receptor type 4 (CXCR-4) per day; 1 to 40 μg/m² plerixafor per day.

Method of administration. In some embodiments, immune checkpoint inhibitors (e.g., PD-1 antagonists) are administered intravenously. Antibodies may also be administered via other modes of administration known in the art. Such modes of administration include inhalation, ingestion, and topical application. Oral administration is also possible for therapeutics, although this form of administration is more challenging for certain biologics such as antibodies. HSCs can be administered through various methods known in the art. In some embodiments, HSCs are administered intravenously (e.g., by intravenous infusion or injection). In some embodiments, the HSC mobilizing agent is administered orally, subcutaneously, intra-muscularly, intravenously, intraventricularly or intrathecally, intraperitoneally, intra-arterially, intravesicularly, or intrapleurally, preferably intravenously.

Timing. In some embodiments, the HSCs are administered as a monotherapy. In some embodiments, an HSC mobilizing agent is administered as a monotherapy. In some embodiments, the HSCs are administered in combination with an HSC mobilizing agent. In some embodiments, the HSCs are administered in combination an immune checkpoint inhibitor (e.g., a PD-1 antagonist). In some embodiments, the HSCs are administered in combination with an HSC mobilizing agent and in further combination with an immune checkpoint inhibitor (e.g., a PD-1 antagonist). In some embodiments, an HSC mobilizing agent is administered in combination with an immune checkpoint inhibitor (e.g., a PD-1 antagonist). When a combination therapy is administered, the HSCs, the HSC mobilizing agent, and/or the immune checkpoint inhibitor (e.g., a PD-1 antagonist) are administered close enough in time to beneficially affect the treatment. In some embodiments, the immune checkpoint inhibitor (e.g., PD-1 antagonist) and/or HSC mobilizing agent is administered separately in time from or simultaneously with the HSCs. In some embodiments, the immune checkpoint inhibitor (e.g., PD-1 antagonist) is administered separately in time from or simultaneously with the HSC mobilizing agent. In some embodiments, the HSCs are administered separately in time from or simultaneously with the HSC mobilizing agent. In some embodiments, the HSCs, the HSC mobilizing agent, and/or the immune checkpoint inhibitor (e.g., PD-1 antagonist) are administered within one day of, within one week of, within one month of, within two months of, within three months of, within four months of, within five months of, or within six months of each other. In some embodiments, the HSCs, the HSC mobilizing agent, and/or the immune checkpoint inhibitor (e.g., PD-1 antagonist) are administered within the same medical visit or procedure. In some embodiments, the immune checkpoint inhibitor (e.g., a PD-1 antagonist) is administered to a subject receiving treatment with HSCs and/or an HSC mobilizing agent. The HSCs, the HSC mobilizing agent, and/or the immune checkpoint inhibitor (e.g., PD-1 antagonist) may be administered in the same or separate compositions. The HSCs, the HSC mobilizing agent, and/or the immune checkpoint inhibitor (e.g., PD-1 antagonist) are administered at the same frequency, or different frequencies.

The present disclosure also contemplates further administering one or more anti-inflammatory agents or antimicrobial agents in combination with HSCs, HSC mobilizing agent and/or immune checkpoint inhibitor (e.g., a PD-1 antagonist). When a combination therapy is administered, the HSCs, the HSC mobilizing agent, the immune checkpoint inhibitor (e.g., a PD-1 antagonist), and/or the anti-inflammatory or antimicrobial agent are administered close enough in time to beneficially affect the treatment. In some embodiments, the anti-inflammatory or antimicrobial agent is administered separately in time from or simultaneously with the immune checkpoint inhibitor (e.g., PD-1 antagonist), HSC mobilizing agent, and/or HSCs. In some embodiments, the anti-inflammatory agent or antimicrobial agent, the HSCs, the HSC mobilizing agent, and/or the immune checkpoint inhibitor (e.g., PD-1 antagonist) are administered within one day of, within one week of, within one month of, within two months of, within three months of, within four months of, within five months of, or within six months of each other. In some embodiments, the anti-inflammatory agent or antimicrobial agent, the HSCs, the HSC mobilizing agent, and/or the immune checkpoint inhibitor (e.g., PD-1 antagonist) are administered within the same medical visit or procedure. In some embodiments, the HSCs, HSC mobilizing agent, and/or the immune checkpoint inhibitor (e.g., a PD-1 antagonist) are administered to a subject receiving treatment with an anti-inflammatory agent or antimicrobial agent. The anti-inflammatory agent or antimicrobial agent, HSCs, the HSC mobilizing agent, and/or PD-1 antagonist may be administered in the same or separate compositions. The anti-inflammatory agent or antimicrobial agent, HSCs, the HSC mobilizing agent, and/or the immune checkpoint inhibitor (e.g., a PD-1 antagonist) are administered at the same frequency, or different frequencies.

The present disclosure also contemplates further administering one or more antiviral agents in combination with HSCs, HSC mobilizing agent and/or PD-1 antagonist administration. When a combination therapy is administered, the HSCs, the HSC mobilizing agent, the immune checkpoint inhibitor (e.g., a PD-1 antagonist), and/or the antiviral agent are administered close enough in time to beneficially affect the treatment. In some embodiments, the antiviral agent is administered separately in time from or simultaneously with the immune checkpoint inhibitor (e.g., PD-1 antagonist), HSC mobilizing agent, and/or HSCs. In some embodiments, the antiviral agent, the HSCs, the HSC mobilizing agent, and/or the immune checkpoint inhibitor (e.g., PD-1 antagonist) are administered within one day of, within one week of, within one month of, within two months of, within three months of, within four months of, within five months of, or within six months of each other. In some embodiments, the antiviral agent, the HSCs, the HSC mobilizing agent, and/or the immune checkpoint inhibitor (e.g., PD-1 antagonist) are administered within the same medical visit or procedure. In some embodiments, the HSCs, HSC mobilizing agent, and/or the immune checkpoint inhibitor (e.g., a PD-1 antagonist) are administered to a subject receiving treatment with an antiviral agent. The antiviral agent, HSCs, the HSC mobilizing agent, and/or PD-1 antagonist may be administered in the same or separate compositions. The antiviral agent, HSCs, the HSC mobilizing agent, and/or the immune checkpoint inhibitor (e.g., a PD-1 antagonist) are administered at the same frequency, or different frequencies.

### T Cell Immunomodulatory Therapy

In another aspect, the present disclosure provides T cell immunomodulatory therapy for coronavirus infections. The inventors have developed a rapidly deployable cellular therapy using a peripheral blood draw to treat coronavirus infections. In one aspect, the disclosure provides methods for treating coronavirus infections by administering to a subject in need thereof, polyclonal T cells (poly-T cells) and SARS-CoV-2-specific T cells (SARS-T cells), in amounts effective to treat the disease. In one aspect, the disclosure provides methods for treating coronavirus infections by administering to a subject in need thereof and receiving anti-viral treatment, poly-T cells and SARS-CoV-2-specific T cells SARS-T cells, in amounts effective to treat the disease. In some embodiments, the poly-T cells and the SARS-T cells are administered in combination with a cytokine (e.g., IL-7, IL-2, IL-15, or IL-21). In some embodiments, the poly-T cells and the SARS-T cells are administered in combination with IL-7. In some embodiments, the coronavirus infection is COVID-19, caused by the novel coronavirus, severe acute respiratory syndrome coronavirus 2 (SARS-CoV-2). In some embodiments, the coronavirus infection is severe COVID-19. In some embodiments, the subject is lymphopenic.

Lymphopenia early after symptomatic presentation has been identified as strongly associated with risk of death as an outcome of infection (Zhou et al., Lancet 395:1054-1062 (2020)). The studies suggest that immune suppression due to loss of adaptive immune surveillance by lymphocytes may be a strong contributing factor to mortality in patients with SARS-Cov-2 infection.

The inventors have recognized that effective immunity can be restored through the adoptive transfer of ex vivo expanded lymphocytes comprising poly-T cells and SARS-CoV-SARS-T cells by adoptive cell therapy (ACT). The inventors have developed methods for the rapid expansion of lymphocytes from a single peripheral blood draw that can be used to rapidly reconstitute immunity in lymphopenic patients with COVID-19 and restore effective immunity. The rapid expansion protocol (REP) for T cell lymphocytes described herein can generate up to a 1000-fold expansion of central and effector memory T cells in 7-14 days from peripheral blood of lymphopenic patients with COVID-19. Without wishing to be bound by theory, it is believed that the co-transfer of polyclonal T cells within the SARS-Cov-2 enriched lymphocytes will provide immune surveillance against other opportunistic infections which take hold during the clinical course of severe COVID-19 infection. Accordingly, the disclosure provides rapidly deployable cellular therapy using a peripheral blood draw to treat coronavirus infections. In some embodiments, the disclosure provides a therapeutic approach for treating lymphopenic patients who are significant risk of death due to COVID-19. A further advantage of this approach is that it would not preclude the administration of other life supporting and anti-viral measures that might be employed in the treatment of patients with COVID-19, but rather is an immune reconstitution effort which is likely required for successful resolution of the infection even with an effective anti-viral treatment.

Adoptive Cell Therapy (ACT or Adoptive Cell Transfer). Adoptive cell therapy is the transfer of cells into a patient for the purpose of transferring immune functionality and other characteristics with the cells. The cells are most commonly immune-derived, for example T cells, and can be autologous or allogeneic. Transfer of autologous cells rather than allogeneic cells minimizes graft versus host disease issues. ACT can be used for treatment of coronavirus infections (e.g., COVID-19). In one aspect, the present disclosure provides methods of treating coronavirus infections (e.g., COVID-19) by ACT of poly-T cells and SARS-T cells. The use of ACT in a subject with a coronavirus infection (e.g., COVID-19) is thought to be advantageous to the subject, with the potential for enhancing immunity. In some embodiments, ACT is used in combination with administration of IL-7, wherein the addition of IL-7 has a synergistic effect. The source of T cells, from which poly-T cells and SARS-T cells are expanded, may be peripheral blood, spleen, or lymph nodes. In some embodiments, the source of T cells is peripheral blood.

In some embodiments, in vivo clonal expansion of SARS-T cells is a biomarker of successful treatment response while failure of T cell clones to expand and persist in the peripheral blood is indicative of treatment failure and disease progression. In some embodiments, an effect of the treatment on the disease is assessed by measuring in vivo clonal expansion of SARS-T cells.

Rapid expansion of lymphocytes. The rapid expansion protocol (REP) for T cell lymphocytes described herein can generate up to a 1000-fold expansion (e.g., up to a 500-fold, up to a 600-fold, up to a 700-fold, up to a 800-fold, up to a 900-fold, or up to a 1000-fold) of central and effector memory T cells in 7-14 days from peripheral blood of lymphopenic patients with COVID-19 (e.g., 7 days, 8 days, 9 days, 10 days, 11 days, 12 days, 13 days, or 14 days), thus providing a rapidly deployable cellular therapy using a peripheral blood draw to treat coronavirus infections. The expansion protocol uses messenger RNA encoding the main structural and immunogenic components of SARS-CoV-2 to function as a substrate for priming antigen-specific T cells and then driving a multi-log-fold expansion of these antigen-specific lymphocytes. SARS-T cells are expanded ex vivo from T cells co-cultured with RNA electroporated antigen-presenting cells expressing one or more SARS-CoV-2 antigens. In some embodiments, the SARS-CoV-2 antigens are polypeptides, or immunogenic fragments thereof, of one or more main structural and immunogenic components of SARS-CoV-2. In some embodiments, the SARS-CoV-2 antigens are polypeptides, or immunogenic fragments thereof, of spike polypeptides, membrane polypeptides, and envelope polypeptides.

In some embodiments, 10⁹ to 10¹¹ T cells can be generated from an input of 10⁷ lymphocytes which can be obtained in a single blood draw, even in patients with severe lymphopenia (CTC Grade 3 or greater). Non-survivors of COVID-19 presented with lymphocyte counts ≤600 cells/μL (CTCAE Criteria 5.0 Grade 2 or greater) while survivors had a mean lymphocyte count >1000 cells/μL (normal) ((Zhou et al., Lancet 395:1054-1062 (2020)). Importantly, these measures were relevant early after symptomatic presentation (Day 4), while peak mortality occurs 14 to 22 days into symptomatic presentation potentially allowing sufficient time to intervene in lymphopenic patients with a rapidly deployable cell therapy. In some embodiments, using a mean of 600 cells cells/μL obtained at Day 4, about 1.2×10⁷ lymphocytes (20 mL blood draw) are expected to be obtained from a single blood draw that can be expanded to about 10⁹-10¹¹ cells. In some embodiments, the cells are expanded to greater than 10⁹ cells, greater than 10¹⁰ cells, or greater than 10¹¹ cells. SARS-Cov-2 antigen-enriched central and effector memory T cells are generated in 7-14 days (e.g., 7 days, 8 days, 9 days, 10 days, 11 days, 12 days, 13 days, or 14 days). In addition to being a personalized cell therapy approach, this process is a scalable treatment modality that is amenable to either a centralized cell therapy manufacturing and distribution model or a point-of-care delivery system in institutions equipped with cell therapy capacity (i.e., bone marrow and stem cell transplantation centers and/or cGMP manufacturing suites).

In some embodiments, the poly-T cells are expanded ex vivo from T cells exposed to anti-CD3 stimulation and a cytokine milieu comprising one or more of IL-2, IL-7, IL-15, and IL-21 to drive the preferential differentiation and expansion of central and effector memory T cells. Different cytokine combinations may be used to achieve a different distribution of effector vs. memory T cells. In some embodiments, the cytokine milieu comprises IL-2. In some embodiments, the cytokine milieu comprises IL-7. In some embodiments, the cytokine milieu comprises IL-15. In some embodiments, the cytokine milieu comprises IL-21. In some embodiments, the cytokine milieu comprises IL-2 in combination with one, two, or three cytokines selected from IL-7, IL-15, and IL-21. In some embodiments, the cytokine milieu comprises IL-7 in combination with one, two, or three cytokines selected from IL-2, IL-15, and IL-21. In some embodiments, the cytokine milieu comprises IL-15 in combination with one, two, or three cytokines selected from IL-2, IL-7, and IL-21. In some embodiments, the cytokine milieu comprises IL-21 in combination with one, two, or three cytokines selected from IL-2, IL-7, and IL-15. Additional cytokines may be added to the cytokine milieu. SARS-CoV-2-specific T cells are expanded ex vivo from T cells stimulated through co-culture with RNA-electroporated antigen-presenting cells expressing one or more SARS-CoV-2 antigens for 3-5 days. The stimulated T cells are then introduced into the rapid expansion protocol and expanded by exposure to anti-CD3 stimulation and a cytokine milieu comprising one or more of IL-2, IL-7, 11-15, and IL-21 to drive the preferential differentiation and expansion of central and effector memory T cells. In some embodiments, the cytokine milieu comprises IL-2. In some embodiments, the cytokine milieu comprises IL-7. In some embodiments, the cytokine milieu comprises IL-15. In some embodiments, the cytokine milieu comprises IL-21. In some embodiments, the cytokine milieu comprises IL-2 in combination with one, two, or three cytokines selected from IL-7, IL-15, and IL-21. In some embodiments, the cytokine milieu comprises IL-7 in combination with one, two, or three cytokines selected from IL-2, IL-15, and IL-21. In some embodiments, the cytokine milieu comprises IL-15 in combination with one, two, or three cytokines selected from IL-2, IL-7, and IL-21. In some embodiments, the cytokine milieu comprises IL-21 in combination with one, two, or three cytokines selected from IL-2, IL-7, and IL-15. Additional cytokines may be added to the cytokine milieu. In some embodiments, the one or more SARS-CoV-2 antigens are polypeptides or immunogenic fragments thereof of the major structural components of the SARS-Cov-2 such as virus membrane, envelope, and/or spike. Rapid expansion protocols are described in Rosenberg et al., J Transl Med. 10:69 (2012).

In one aspect, the disclosure provides a method for making SARS-CoV-2-specific T cells, comprising: a) contacting antigen-presenting cells with one or more mRNAs encoding at least one SARS-CoV-2 antigen, in vitro under a condition sufficient for the at least one SARS-CoV-2 antigen to be presented by the antigen-presenting cells; and b) contacting lymphocytes with the antigen-presenting cells of step a) under conditions sufficient to produce the lymphocytes, wherein the lymphocytes are capable of eliciting an immune response against a cell that expresses a SARS-CoV-2 antigen.

Antigen presenting cells. Stimulation of lymphocytes with antigen presenting cells presenting SARS-CoV-2 antigens will lead to an enrichment of SARS-CoV-2 specific T cells that can be rapidly expanded for adoptive T cells transfers. In some embodiments, the antigen presenting cells are T cells, dendritic cells, or peripheral blood mononuclear cells. In some embodiments, the antigen presenting cells are peripheral blood mononuclear cells.

Additional cytokines. In one aspect, the present disclosure posits that combining adoptive cell therapy with the administration of one or more cytokines selected from IL-7, IL-2, IL-15 and IL-21 to the subject enhances treatment efficacy in a subject having a coronavirus infection (e.g., COVID-19). Without wishing to be bound by theory, it is believed that the engraftment, expansion, and persistence of adoptively transferred lymphocytes may be potentiated by the administration of one or more of IL-7, IL-2, IL-15 and IL-21. Synergistic interaction of adoptive cell transfer of poly-T cells and SARS-T cells with administration of one or more of IL-7, IL-2, IL-15, and IL-21 may potentiate the effects of this treatment, leading to effective immune reconstitution.

In some embodiments, the cytokine administered is IL-7. In some embodiments, the IL-7 is recombinant human IL-7. In some embodiments, the recombinant human IL-7 is CYT107 (RevImmune). In some embodiments, the cytokine administered is IL-2. In some embodiments, the cytokine administered is IL-15. In some embodiments, the cytokine administered is IL-21. In some embodiments, two, three, or four cytokines selected from IL-7, IL-2, IL-15, and IL-21 are administered. In some embodiments, IL-7 is administered in combination with one, two, or three cytokines selected from IL-2, IL-15, and IL-21. In some embodiments, IL-2 is administered in combination with one, two, or three cytokines selected from IL-7, IL-15, and IL-21. In some embodiments, IL-15 is administered in combination with one, two, or three cytokines selected from IL-2, IL-7, and IL-21. In some embodiments, IL-21 is administered in combination with one, two, or three cytokines selected from IL-2, IL-7, and IL-15.

Antiviral agents. The present disclosure contemplates further administering one or more antiviral agents in combination with ACT and/or cytokine (e.g., IL-7, IL-2, IL-12, or IL-15) administration. The one or more viral agents may include, but are not limited to, lopanivir, ritonavir, and remdesivir.

Dosages. In some embodiments, an effective amount of poly-T cells is about 10⁵-10¹⁰ cells/kg. In some embodiments, an effective amount of poly-T cells is about 10⁵ cells/kg, about 10⁶ cells/kg, about 10⁷ cells/kg, about 10⁸ cells/kg, about 10⁹ cells/kg, or about 10¹⁰ cells/kg.

In some embodiments, an effective amount of SARS-T cells is about 10⁵-10¹⁰ cells/kg. In some embodiments, an effective amount of poly-T cells is about 10⁵ cells/kg, about 10⁶ cells/kg, about 10⁷ cells/kg, about 10⁸ cells/kg, about 10⁹ cells/kg, or about 10¹⁰ cells/kg.

Method of administration. In some embodiments, adoptive transfer of poly-T cells and SARS-T cells is administered intravenously (e.g., by infusion or injection). The T cells may also be administered through various methods known in the art. In some embodiments, the administration of IL-17 is intravenous. IL-7 may also be administered via other modes of administration known in the art.

Timing. In some embodiments, adoptive cell transfer of poly-T cells and SARS-T cells is combined with the administration of one or more cytokines (e.g., IL-7, IL-2, IL-15, or IL-21) and/or administration of one or more antiviral agents. When a combination therapy is administered, poly-T cells and SARS-T cells, one or more cytokines (e.g., IL-7, IL-2, IL-15, or IL-21), and/or one or more antiviral agents are administered close enough in time to beneficially affect the treatment. In some embodiments, one or more cytokines (e.g., IL-7, IL-2, IL-15, or IL-21) and/or one or more antiviral agents are administered separately in time from or simultaneously with the HSCs. In some embodiments, poly-T cells and SARS-T cells, one or more cytokines (e.g., IL-7, IL-2, IL-15, or IL-21), and/or one or more antiviral agents are administered within one day of, within one week of, within one month of, within two months of, within three months of, within four months of, within five months of, or within six months of each other. In some embodiments, the poly-T cells and SARS-T cells, one or more cytokines (e.g., IL-7, IL-2, IL-15, or IL-21), and/or one or more antiviral agents are administered within the same medical visit or procedure. In some embodiments, the poly-T cells and SARS-T cells, and/or one or more cytokines (e.g., IL-7, IL-2, IL-15, or IL-21) are administered to a subject receiving treatment with one or more antiviral agents. The poly-T cells and SARS-T cells, one or more cytokines (e.g., IL-7, IL-2, IL-15, or IL-21), and/or one or more antiviral agents may be administered in the same or separate compositions. The poly-T cells and SARS-T cells, one or more cytokines (e.g., IL-7, IL-2, IL-15, or IL-21), and/or one or more antiviral agents are administered at the same frequency, or different frequencies.

### EXAMPLES

### Example 1. Use of Hematopoietic Stem Cells (HSCs) in Combination with Immune Checkpoint Blockade for Treating Severe COVID-19

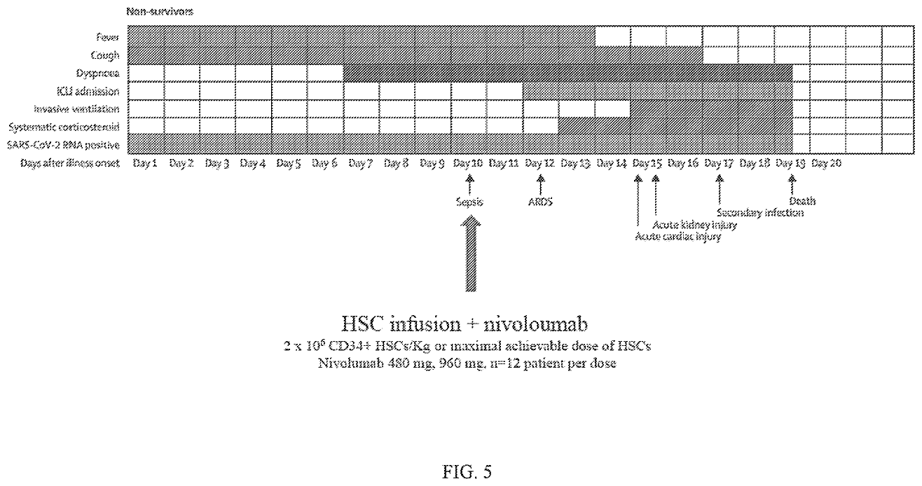

The purpose of this study is to determine whether the combination of HLA-matched allogenic hematopoietic stem cell (HSC) transfer with PD-1 blockade is safe, feasible, and leads to enhanced reconstitution of T cell immunity in patients with severe COVID-19 infection. A single injection of HLA-matched CD34+ HSCs from umbilical cord blood combined with a single injection of nivolumab (480 mg or 960 mg) is administered to a patient with severe COVID-19 infection and associated lymphopenia (FIG. 5).

The effects of HSC transfer+nivolumab on immune reconstitution (lymphopenia and T cell function) in patients with severe COVID-19 are examined. Other observed metrics include clinical outcomes such as days in hospital and/or ICU, and overall survival; viral clearance by quantitative PCR; antibody signatures pre and post therapy; and peripheral lymphocyte phenotype changes, TCR clonotypic gene analysis, and gene expression analysis.

The combination of HSC transfer+PD-1 blockade leads to the attenuation of inflammatory toxicity, rapid reconstitution of T cell immunity, and viral clearance in lymphopenic patients with severe COVID-19.

### Example 2. Use of Adoptive Cell Transfer (ACT) for Treating Severe COVID-19

The purpose of this study is to determine whether the administration of polyclonal T-cells (poly-T cells) and SARS-CoV-2-specific T cells (SARS-T cells) is safe, feasible, and leads to immune reconstitution in patients with severe COVID-19 infection.

20-30 mLs of peripheral blood are drawn twice weekly for expansion of poly-T cells and SARS-CoV-2-specific T cells for intravenous delivery starting 7 days from blood draw. Poly-T cells with global specificity are immediately expanded by isolation of T cells using gradient separation and placed into the rapid expansion protocol. The rapid expansion protocol uses anti-CD3 stimulation and a cytokine milieu containing IL-2, IL-7, and IL-21 that drives preferential differentiation and expansion of central and effector memory T cells. SARS-CoV-2-specific T cells are stimulated through co-culture with RNA-electroporated mononuclear cells expressing the major structural components of the SARS-Cov-2 virus membrane, envelope, and spike for 3-5 days and then entry of stimulated T cells into the rapid expansion protocol. Thus, SARS-Cov-2-specific T cells are expected to be ready for infusion within 10 to 14 days of blood draw.

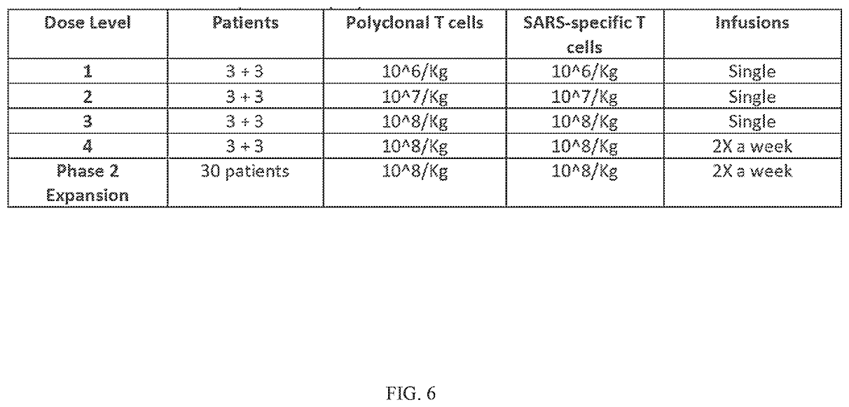

The patients are treated in accordance with the treatment schemes shown in FIG. 6, with poly-T cells and SARS-T cells administered at 106/kg each, 107/kg, or 108/kg each, once or twice a week.

The effects of adoptive cell transfer of poly-T cells and SARS-T cells are examined. Pre- and post-treatment antibodies to SARS-CoV-2 and baseline and post-infusion T cell responses to the SARS-CoV-2 structural components are also measured. T cell responses will be measured by Elispot, Cytokine Bead Array analysis of T cells simulated by SARS-CoV-2 antigens, TCR sequencing and single cell RNA sequencing of peripheral blood mononuclear cells before and after adoptive T cell therapy. The in vivo clonal expansion of SARS-CoV-2-specific T cells after adoptive transfer is a biomarker of successful treatment response. TCR clonal expansion will be examined by T cell RNA sequencing to determine if expansion of SARS-CoV-2-specific T cells is associated with favorable treatment response. Single cell RNA sequencing will identify genotypic profiles of cells associated with viral clearance and resolution of disease in treated patients.

Adoptive transfer of rapidly expanded polyclonal and SARS-Cov-2-specific memory T cells from lymphopenic patients with COVID-19 provides rapid reconstitution of immune surveillance capabilities against SARS-CoV-2 infections and reduces the morbidity, mortality, and duration of severe COVID-19.

### Embodiments

In some embodiments, the present disclosure provides:

1. A method for treating COVID-19 in a subject having COVID-19, comprising administering to the subject hematopoietic stem cells in an amount effective to treat the disease.

2. The method of embodiment 1, further comprising administering a programmed death 1 (PD-1) antagonist.

3. The method of embodiment 2, wherein the PD-1 antagonist is an agent that binds to and antagonizes programmed death 1 (PD-1).

4. The method of embodiment 3, wherein the agent that binds to and antagonizes PD-1 is a peptide that binds PD-1.

5. The method of embodiment 3, wherein the agent that binds to and antagonizes PD-1 is a humanized antibody that selectively binds PD-1.

6. The method of embodiment 5, wherein the humanized antibody that selectively binds PD-1 is nivolumab, pembrolizumab, pidilizumab, MEDI-0680, REGN2810, or AMP-224.

7. The method of embodiment 6, wherein the humanized antibody that selectively binds PD-1 is nivolumab.

8. The method of any preceding embodiment, wherein the method further comprises administering to the subject a hematopoietic stem cell mobilizing agent.

9. The method of embodiment 8, wherein the mobilizing agent is granulocyte colony-stimulating factor (G-CSF), PEGylated G-CSF (pegfilgratism), lenogratism, a glycosylated form of G-CSF, C-X-C motif chemokine 2 (CXCL2), C-X-C chemokine receptor type 4 (CXCR-4), or plerixafor.

10. The method of any one of embodiments 2-9, wherein the PD-1 antagonist is administered separately in time from or simultaneously with the hematopoietic stem cells.

11. The method of any preceding embodiment, wherein the PD-1 antagonist is administered at the same time as, within one day of, within one week of, within one month of, within two months of, within three months of, within four months of, within five months of, or within six months of administering the hematopoietic stem cells.

12. The method of embodiment 8 or 9, wherein the PD-1 antagonist is administered at the same time as, within one day of, within one week of, within one month of, within two months of, within three months of, within four months of, within five months of, or within six months of administering the hematopoietic stem cell mobilizing agent.

13. The method of any one of embodiments 2-12, wherein the PD-1 antagonist is administered intravenously or subcutaneously.

14. The method of any preceding embodiment, wherein the hematopoietic stem cells are administered intravenously.

15. The method of any one of embodiments 8, 9, or 12-14, wherein the hematopoietic stem cell mobilizing agent is administered intravenously, intradermally, or subcutaneously.

16. The method of any preceding embodiment, wherein the source of hematopoietic stem cells is bone marrow, bone marrow lineage depleted cells (lin−), cKit+ purified lineage negative bone marrow derived cells, Sca+ purified lineage negative bone marrow derived cells, cKit+Sca+ purified bone marrow derived cells, mobilized from host bone marrow using GM-CSF, G-CSF, mobilized from host bone marrow using AMD3100, Plerixafor, or the molecule 1,1′-[1,4-phenylenebis(methylene)]bis [1,4,8,11-tetraazacyclotetradecane], umbilical cord blood or cord-blood derived stem cells, unselected umbilical cord blood stem cells (UCBSCs) or UCBSCs selected for CD34+ cells, CCR2+, or lin(−) cells, human leukocyte antigen (HLA)-matched blood, mesenchymal stem cells derived from blood or marrow, hematopoietic stem cells differentiated from induced pluripotent stem cells, mobilized peripheral blood, peripheral blood, hematopoietic stem cell subsets including lin− cells purified with CCR2+ marker, lineage negative purified peripheral blood, or CD34+ enriched peripheral blood.

17. The method of any preceding embodiment, wherein the source of hematopoietic stem cells is bone marrow, peripheral blood, umbilical cord blood, or induced pluripotent stem cells.

18. The method of any preceding embodiment, wherein the source of hematopoietic stem cells is autologous.

19. The method of any one of embodiments 1-17, wherein the source of hematopoietic stem cells is allogeneic and the donor cells are HLA-matched to the recipient.

20. The method of any one of embodiments 1-18, wherein a sample containing the hematopoietic stem cells is obtained from the subject and processed to expand the number of stem cells within the sample, in vitro, prior to administering to the subject the hematopoietic stem cells.

21. The method of any one of embodiments 1-18, wherein a sample containing the hematopoietic stem cells is obtained from the subject and processed to increase the percentage of stem cells within the sample, in vitro, prior to administering to the subject the hematopoietic stem cells.

22. The method of any preceding embodiment, wherein at least 5, 10, 15, 20, 25, 30, 35, 40, 45, or 50 percent of the hematopoietic stem cells are CCR2 positive (CCR2+), CD34 positive (CD34+), and/or lineage negative (lin−) cells.

23. The method of any preceding embodiment, wherein the hematopoietic stem cells for administration to the subject are enriched ex vivo for CCR2 positive (CCR2+) cells, for CD34 positive (CD34+) cells and/or for lineage negative (lin−) cells prior to administration to the subject.

24. The method of any preceding embodiment, wherein the hematopoietic stem cells are processed ex vivo to deplete CCR2 negative (CCR2−) cells before administration to the subject.

25. The method of any preceding embodiment, wherein the COVID-19 is severe COVID-19.

26. A method for treating COVID-19 in a subject having COVID-19, comprising administering to the subject a hematopoietic stem cell mobilizing agent in an amount effective to treat the disease.

27. The method of embodiment 26, wherein the mobilizing agent is granulocyte colony-stimulating factor (G-CSF), PEGylated G-CSF (pegfilgratism), lenogratism, a glycosylated form of G-CSF, C-X-C motif chemokine 2 (CXCL2), C-X-C chemokine receptor type 4 (CXCR-4), or plerixafor.

28. The method of embodiment 26 or 27, further comprising administering a programmed death 1 (PD-1) antagonist.

29. The method of any one of embodiments 26-28, further comprising administering hematopoietic stem cells.

30. A method for treating COVID-19 in a subject having COVID-19 comprising, administering a hematopoietic stem cell mobilizing agent to the subject, harvesting hematopoietic stem cells from the subject, enriching the harvested stem cells for CCR2 positive (CCR2+), CD34 positive (CD34+), or lineage negative (lin−) cells, optionally depleting the harvested stem cells or CCR2− cells, administering to the subject the enriched harvested stem cells, and administering to the subject a PD-1 antagonist.

31. A method for treating COVID-19 in a subject having COVID-19, comprising administering to the subject polyclonal T cells (poly-T cells) and SARS-CoV-2-specific T cells (SARS-T cells), in amounts effective to treat the disease.

32. The method of embodiment 31, wherein the poly-T cells and the SARS-T cells are autologous.

33. The method of embodiment 31 or 32, wherein the source of T cells is peripheral blood, spleen, or lymph nodes.

34. The method of embodiment 33, wherein the source of T cells is peripheral blood.

35. The method of any one of embodiments 31-34, wherein the poly-T cells are expanded ex vivo from T cells exposed to anti-CD3 stimulation and a cytokine milieu comprising one or more of IL-2, IL-7, IL-15, and IL-21, to drive the preferential differentiation and expansion of central and effector memory T cells.

36. The method of any one of embodiments 31-35, wherein the SARS-T cells are expanded ex vivo from T cells co-cultured with RNA electroporated antigen presenting cells expressing a SARS-CoV-2 antigen.

37. The method of embodiment 36, wherein the antigen presenting cells are dendritic cells, T cells, or peripheral blood mononuclear cells.

38. The method of embodiment 37, wherein the antigen presenting cells are peripheral blood mononuclear cells.

39. The method of any one of embodiments 36-38, wherein the SARS-CoV-2 antigen is a polypeptide, or an immunogenic fragment thereof, selected from the group consisting of: spike polypeptides, membrane polypeptides, and envelope polypeptides.

40. The method of any one of embodiments 31-39, wherein the COVID-19 is severe COVID-19.

41. The method of any one of embodiments 31-40, further comprising administering one or more antiviral agents.

42. The method of embodiment 41, wherein the one or more antiviral agents are selected from lopanivir, ritonavir, and remdesivir.

43. The method of embodiment 41 or 42, wherein the one or more antiviral agents are administered separately in time from or simultaneously with the poly-T cells and/or the SARS-T cells.

44. The method of any one of embodiments 41-43, wherein the one or more antiviral agents are administered at the same time as, within one day of, within one week of, within one month of, within two months of, within three months of, within four months of, within five months of, or within six months of administering the poly-T cells and/or the SARS-T cells.

45. The method of any one of embodiments 31-44, wherein an effect of the treatment on the disease is assessed by measuring clonal expansion of SARS-T cells.

46. A method for treating COVID-19 in a subject having COVID-19 and receiving antiviral treatment, comprising administering to the subject polyclonal T cells (poly-T cells) and SARS-CoV-2-specific T cells (SARS-T cells), in amounts effective to treat the disease.

47. The method of embodiment 46, wherein the antiviral treatment comprises administration of one or more of lopanivir, ritonavir, and remdesivir.

### Equivalents and Scope

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents, and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B,” the disclosure also contemplates the alternative embodiments “a composition consisting of A and B” and “a composition consisting essentially of A and B.”

What is claimed is:

1. A method for treating COVID-19 in a subject in need thereof, comprising administering to the subject hematopoietic stem cells in an amount effective to treat the disease.

2. The method of claim 1, further comprising administering a programmed death 1 (PD-1) antagonist.

3. The method of claim 2, wherein the PD-1 antagonist is an agent that binds to and antagonizes programmed death 1 (PD-1).

4. The method of claim 3, wherein the agent that binds to and antagonizes PD-1 is a peptide that binds PD-1.

5. The method of claim 3, wherein the agent that binds to and antagonizes PD-1 is a humanized antibody that selectively binds PD-1.

6. The method of claim 5, wherein the humanized antibody that selectively binds PD-1 is nivolumab, pembrolizumab, pidilizumab, MEDI-0680, REGN2810, or AMP-224.

7. The method of claim 6, wherein the humanized antibody that selectively binds PD-1 is nivolumab.

8. The method of any preceding claim, wherein the method further comprises administering to the subject a hematopoietic stem cell mobilizing agent.

9. The method of claim 8, wherein the mobilizing agent is granulocyte colony-stimulating factor (G-CSF), PEGylated G-CSF (pegfilgratism), lenogratism, a glycosylated form of G-CSF, C-X-C motif chemokine 2 (CXCL2), C-X-C chemokine receptor type 4 (CXCR-4), or plerixafor.

10. The method of any one of claims 2-9, wherein the PD-1 antagonist is administered separately in time from or simultaneously with the hematopoietic stem cells.

11. The method of any preceding claim, wherein the PD-1 antagonist is administered at the same time as, within one day of, within one week of, within one month of, within two months of, within three months of, within four months of, within five months of, or within six months of administering the hematopoietic stem cells.

12. The method of claim 8 or 9, wherein the PD-1 antagonist is administered at the same time as, within one day of, within one week of, within one month of, within two months of, within three months of, within four months of, within five months of, or within six months of administering the hematopoietic stem cell mobilizing agent.

13. The method of any one of claims 2-12, wherein the PD-1 antagonist is administered intravenously or subcutaneously.

14. The method of any preceding claim, wherein the hematopoietic stem cells are administered intravenously.

15. The method of any one of claim 8, 9, or 12-14, wherein the hematopoietic stem cell mobilizing agent is administered intravenously, intradermally, or subcutaneously.

16. The method of any preceding claim, wherein the source of hematopoietic stem cells is bone marrow, bone marrow lineage depleted cells (lin−), cKit+ purified lineage negative bone marrow derived cells, Sca+ purified lineage negative bone marrow derived cells, cKit+Sca+ purified bone marrow derived cells, mobilized from host bone marrow using GM-CSF, G-CSF, mobilized from host bone marrow using AMD3100, Plerixafor, or the molecule 1,1′-[1,4-phenylenebis(methylene)]bis [1,4,8,11-tetraazacyclotetradecane], umbilical cord blood or cord-blood derived stem cells, unselected umbilical cord blood stem cells (UCBSCs) or UCBSCs selected for CD34+ cells, CCR2+, or lin(−) cells, human leukocyte antigen (HLA)-matched blood, mesenchymal stem cells derived from blood or marrow, hematopoietic stem cells differentiated from induced pluripotent stem cells, mobilized peripheral blood, peripheral blood, hematopoietic stem cell subsets including lin− cells purified with CCR2+ marker, lineage negative purified peripheral blood, or CD34+ enriched peripheral blood.

17. The method of any preceding claim, wherein the source of hematopoietic stem cells is bone marrow, peripheral blood, umbilical cord blood, or induced pluripotent stem cells.

18. The method of any preceding claim, wherein the source of hematopoietic stem cells is autologous.

19. The method of any one of claims 1-17, wherein the source of hematopoietic stem cells is allogeneic and the donor cells are HLA-matched to the recipient.

20. The method of any one of claims 1-18, wherein a sample containing the hematopoietic stem cells is obtained from the subject and processed to expand the number of stem cells within the sample, in vitro, prior to administering to the subject the hematopoietic stem cells.

21. The method of any one of claims 1-18, wherein a sample containing the hematopoietic stem cells is obtained from the subject and processed to increase the percentage of stem cells within the sample, in vitro, prior to administering to the subject the hematopoietic stem cells.

22. The method of any preceding claim, wherein at least 5, 10, 15, 20, 25, 30, 35, 40, 45, or 50 percent of the hematopoietic stem cells are CCR2 positive (CCR2+), CD34 positive (CD34+), and/or lineage negative (lin−) cells.

23. The method of any preceding claim, wherein the hematopoietic stem cells for administration to the subject are enriched ex vivo for CCR2 positive (CCR2+) cells, for CD34 positive (CD34+) cells and/or for lineage negative (lin−) cells prior to administration to the subject.

24. The method of any preceding claim, wherein the hematopoietic stem cells are processed ex vivo to deplete CCR2 negative (CCR2−) cells before administration to the subject.

25. The method of any preceding claim, wherein the COVID-19 is severe COVID-19.

26. A method for treating COVID-19 in a subject in need thereof, comprising administering to the subject a hematopoietic stem cell mobilizing agent in an amount effective to treat the disease.

27. The method of claim 26, wherein the mobilizing agent is granulocyte colony-stimulating factor (G-CSF), PEGylated G-CSF (pegfilgratism), lenogratism, a glycosylated form of G-CSF, C-X-C motif chemokine 2 (CXCL2), C-X-C chemokine receptor type 4 (CXCR-4), or plerixafor.

28. The method of claim 26 or 27, further comprising administering a programmed death 1 (PD-1) antagonist.

29. The method of any one of claims 26-28, further comprising administering hematopoietic stem cells.

30. A method for treating COVID-19 in a subject in need thereof, comprising administering a hematopoietic stem cell mobilizing agent to the subject, harvesting hematopoietic stem cells from the subject, enriching the harvested stem cells for CCR2 positive (CCR2+), CD34 positive (CD34+), or lineage negative (lin−) cells, optionally depleting the harvested stem cells or CCR2− cells, administering to the subject the enriched harvested stem cells, and administering to the subject a PD-1 antagonist.
